(19) United States
(12) Patent Application Publication
(10) Pub. No.: US 2010/0234697 A1
(43) Pub. Date: Sep. 16, 2010

(54) SYSTEMS, DEVICES, AND METHODS FOR MONITORING A SUBJECT

(75) Inventors: Timothy J. Walter, Upper Arlington, OH (US); Uma Marar, Blacklick, OH (US)

Correspondence Address: MUETING, RAASCH & GEBHARDT, P.A., P.O. BOX 581336, MINNEAPOLIS, MN, 55458-1336, US

(73) Assignee: Lotus Magnus, LLC, Grove City, OH (US)

(21) Appl. No.: 12/438,273

(22) PCT Filed: Apr. 29, 2008
(86) PCT No.: PCT/US08/61889

§ 371 Date: Feb. 20, 2009

(51) Int. Cl. A61B 5/0476; A61B 5/0488; A61B 5/0496

(52) U.S. Cl. 600/301

(57) ABSTRACT

Systems, devices, and methods for monitoring a subject using a monitoring patch that may include electroencephalography apparatus, electromyography apparatus, and electrooculography apparatus.

The present invention relates generally to systems, devices, and methods for monitoring a subject (e.g., monitoring a subject's various states of sleep and wakefulness). More specifically, the present invention relates to systems, devices, and methods that may monitor the neural, muscular, and/or ocular activity of a subject with a small portable device to determine the amount and/or quality of the sleep the subject undergoes and/or the vigilance of the subject while the subject is awake.

Electroencephalography (EEG) records the neural activity of electrical potential across cell membranes, which are detected through the cerebral cortex and recorded by a plurality of electrodes. The changes in electrical potential in the cortex contain rhythmical activity, which typically occur at frequencies of about 0.5 to about 70 cycles per second (hertz). While awake, fast, random signals are predominately generated at low voltage and mixed frequency. While asleep, more predictable signals are generated at a low voltage and predictable frequencies over predictable periods.

Five distinct brain wave patterns that are commonly detected during an EEG recording are delta waves (e.g., about 0.5-3 hertz), theta waves (e.g., about 3-8 hertz), alpha waves (e.g., about 8-12 hertz), beta waves (e.g., about 13-20 hertz), and gamma waves (e.g., about 26-70 hertz). Many of these frequencies may be observed in a subject's sleep cycle. A sleep cycle may be defined as a progression of brainwave patterns that may be seen while a subject is sleeping. Generally, subjects undergo several sleep cycles per night, each lasting around ninety minutes. Each progression of brainwave patterns during the sleep cycle may be referred to as a stage of the sleep cycle. Generally, each sleep cycle progresses consecutively through stage I sleep, stage II sleep, stage III sleep, stage IV sleep (stage III sleep and stage IV sleep may be grouped together and refereed to as slow wave sleep), briefly back to stage II sleep, and then rapid eye movement (REM) sleep.

Waking consciousness is generally experienced neurophysiologically at a brainwave frequency of about forty hertz.

Electrooculography (EOG) records the ocular activity of the electrical potential from the retina, which consists of an electrically-charged nerve membrane. EOG signals can be measured by placing electrodes near an eye. Motion of an eye may cause a measurable change of electrical potential between two or more surface electrodes.

Electromyography (EMG) records the muscular activity of electrical potential across muscular membranes, which range between about 50 microvolts to about 30 millivolts (depending on the muscle under observation). Typical repetition rate of muscle unit firing is about 7 hertz to about 20 hertz, depending on the size of the muscle, the type of muscle, etc. EMG signals may be recorded within a muscle (i.e., intramuscular EMG) or on the surface a subject's skin outside of a muscle.

Sleep may be characterized by specific patterns in a subject's EEG and/or EMG. Analysis of EEG and/or EMG recordings may be performed to, e.g., diagnose various sleep disorders such as, circadian rhythm disorders (e.g., advanced sleep phase syndrome, delayed sleep phase syndrome, free-running type, jet lag, and shift work sleep disorder), disorders of REM sleep (e.g., REM Sleep Behavior Disorder). Further, analysis of EEG and/or EMG recordings may be performed to calculate the amount of sleep a subject obtains in regards to insomnia (e.g., inadequate sleep hygiene, paradoxical insomnia, primary insomnia, secondary insomnia, psychophysiological insomnia) in a way that would be more objective and more accurate than the currently used modalities of actigraphy and/or a sleep diary, hypersomnia (narcolepsy, idiopathic hypersomnia, Klein-Levin Syndrome, and menstrual related hypersomnia) or to measure the effects of sleep promoting and alertness promoting pharmaceuticals on the state of vigilance of the subject, etc.

Sleep onset is characterized by specific changes in a subject's EEG and/or EMG data. As such, signal data recorded by EEG and/or EMG apparatus may be utilized to determine how long an individual subject has slept. For example, a skilled practitioner may analyze the data for patterns of sleep and wakefulness that would provide diagnostic support for the various sleep and wakefulness disorders described herein. The data would be analyzed to determine how long a subject has slept. Also, for example, a computer may analyze the data using pre-existing algorithms and software (e.g., Polysmith 2003) to score the various stages of sleep and wakefulness to determine how long a subject as slept.

The determination of how long an individual subject has slept over a given or selected time period has a number of commercial applications. Such a determination may be important for shift workers, truck drivers, train operators, air traffic controllers, airplane pilots, and other subjects whose work could be dangerous if they become too drowsy. In addition, many of these workers and others may be required by governmental entities (e.g., the National Transportation Safety Board or the Federal Motor Carrier Administration), worker unions, employers, etc. to sleep a minimum number of hours per work week.

Sleep may be an important factor in determining vigilance of a subject. Vigilance may be characterized by specific changes in a subject's EEG and/or EMG data. As such, signal data recorded by EEG and/or EMG may be utilized to determine the vigilance of a subject. Such signal data may be monitored in real-time to determine the vigilance of a subject. For example, devices exist that analyze EEG or EMG data to determine if a truck driver is becoming drowsy, and subsequently alerts the driver to increase his/her vigilance (e.g., using an alarm). Such data may also be recorded and then analyzed offline.

Further, collecting EEG and/or EMG data is useful for various sleep disorder testing. For example, EEG and/or EMG data may be collected during a Multiple Sleep Latency Test (MSLT). Typically, a MSLT is conducted the day following an overnight sleep. The purpose of this test is to objectively measure daytime sleepiness and to look for REM sleep during daytime naps. Although REM sleep may be seen in normal subjects during the day under special circumstances, often, REM sleep during day is indicative of narcolepsy (i.e., a disorder of REM sleep).

Also further, for example, collecting EEG data is useful in determining the duration and/or timing of sleep periods in patients with circadian rhythm disorders and insomnia. Often, circadian rhythm disorders and insomnia are determined by actigraphy and/or sleep logs, which may be inherently inaccurate.

Still further, for example, EEG and/or EMG data may be collected during a Maintenance of Wakefulness Test (MWT). The MWT is similar to the MSLT except that it is usually performed after the subject as been treated for a sleep disorder. During the MWT, a subject will stay awake during the recording sessions to demonstrate that the subject no longer has excessive daytime sleepiness. The MWT is commonly performed for truck drivers, pilots, or people operating heavy machinery and may be required on a yearly basis, depending on the requirements dictated by employers and/or by governmental guidelines. Currently, both the MSLT and MWT are routinely performed in accredited sleep diagnostic testing facilities and sleep centers.

### SUMMARY OF THE INVENTION

In one aspect, the present invention provides a system for monitoring the neural, muscular, and ocular activity of a subject. The system may include a self-contained monitoring patch and an analysis system to analyze the neural activity data, the muscular activity data, and the ocular activity data. The monitoring patch may include: electroencephalography apparatus to monitor neural activity of the subject during a selected time period; electromyography apparatus to monitor muscular activity of the subject during the selected time period; electrooculography apparatus to monitor ocular activity of the subject during a selected period of time; storage apparatus electrically coupled to the electroencephalography apparatus to store neural activity data, to the electromyography apparatus to store muscular activity data, and to the electrooculography apparatus to store ocular activity data; a power supply; and adhesive to attach the monitoring patch to the subject. Each of the electroencephalography apparatus, the electromyography apparatus, and the electrooculography apparatus may include an electrode The analysis system may include: an input interface coupleable to the storage apparatus to receive the neural activity data, the muscular activity data, and the ocular activity data; processing apparatus coupled to the input interface to analyze the neural activity data, the muscular activity data, and the ocular activity data; and an output interface coupled to the processing apparatus to output results from the analysis of the neural activity data, the muscular activity data, and the ocular activity data.

In another aspect, the present invention provides a method for monitoring the neural, muscular, and ocular activity of a subject. The method may include: providing a self-contained monitoring patch; attaching the monitoring patch to a subject; monitoring neural activity of the subject with the electroencephalography apparatus during a selected period of time to obtain neural activity data; monitoring muscular activity of the subject with the electromyography apparatus during a selected period of time to obtain muscular activity data; monitoring ocular activity of the subject with the electrooculography apparatus during a selected period of time to obtain ocular activity data; transferring the neural activity data, the muscular activity data, and the ocular activity data from the storage apparatus of the monitoring patch to an analysis system; analyzing the neural activity data, the muscular activity data, and the ocular activity data using the analysis system; and outputting a result based on the analysis of the neural activity data, the muscular activity data, and the ocular activity data. The monitoring patch may include: electroencephalography apparatus electrically coupled to a storage apparatus; electromyography apparatus electrically coupled to the storage apparatus; electrooculography apparatus electrically coupled to the storage apparatus; one or more electrodes electrically coupled to the electroencephalography apparatus, the electromyography apparatus, and the electrooculography apparatus; a power supply; and adhesive.

In yet another aspect, the present invention provides a monitoring patch for monitoring the neural, muscular, and ocular activity of a subject. The monitoring patch may include: electroencephalography apparatus to monitor neural activity of the subject during a selected time period; electromyography apparatus to monitor muscular activity of the subject during a selected time period; electrooculography apparatus to monitor ocular activity of the subject during a selected period of time; storage apparatus electrically coupled to the electroencephalography apparatus to store neural activity data, to the electromyography apparatus to store muscular activity data, and to the electrooculography apparatus to store ocular activity data; power supply; and adhesive to attach the monitoring patch to the subject. Each of the electroencephalography apparatus, the electromyography apparatus, and the electrooculography apparatus may include an electrode. Further, the monitoring patch may be self-contained.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE DRAWING

FIG. 1A is an illustrative view of one exemplary embodiment of a monitoring patch according to the present invention.

FIG. 1B is an illustrative view of one exemplary embodiment of a monitoring patch according to the present invention.

FIG. 2A is a view of one exemplary embodiment of a monitoring patch according to the present invention.

FIG. 2B is another view of the exemplary embodiment of the monitoring patch of FIG. 2A according to the present invention.

FIG. 2C is a view of one exemplary embodiment of a monitoring according to the present invention.

FIG. 2D is a view of one exemplary embodiment of a monitoring according to the present invention.

FIG. 3 is a diagrammatic representation of one exemplary embodiment of a monitoring patch according to the present invention.

FIG. 4 is a diagrammatic representation of one exemplary embodiment of an analysis system according to the present invention.

FIG. 5 is a diagrammatic representation of one exemplary embodiment of a monitoring and analysis system according to the present invention.

FIG. 6 is a flow chart of one exemplary method of monitoring a subject's neural activity according to the present invention.

FIG. 7 is a flow chart of one exemplary method of monitoring a subject's muscular activity according to the present invention.

FIG. 8 is a flow chart of one exemplary method of monitoring a subject's neural and muscular activity according to the present invention.

FIG. 9 is a flow chart of one exemplary method of monitoring a subject's neural, muscular, and ocular activity according to the present invention.

FIG. 10 is an illustrative view of one exemplary output report that may be provided by the methods and/or systems according to the present invention.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention. Unless stated otherwise herein, the figures of the drawing are rendered primarily for clarity and thus may not be drawn to scale.

As used herein, “a,” “an,” “the,” “at least one,” and “one or more” are used interchangeably. The term “and/or” (if used) means one or all of the listed elements or a combination of any two or more of the listed elements.

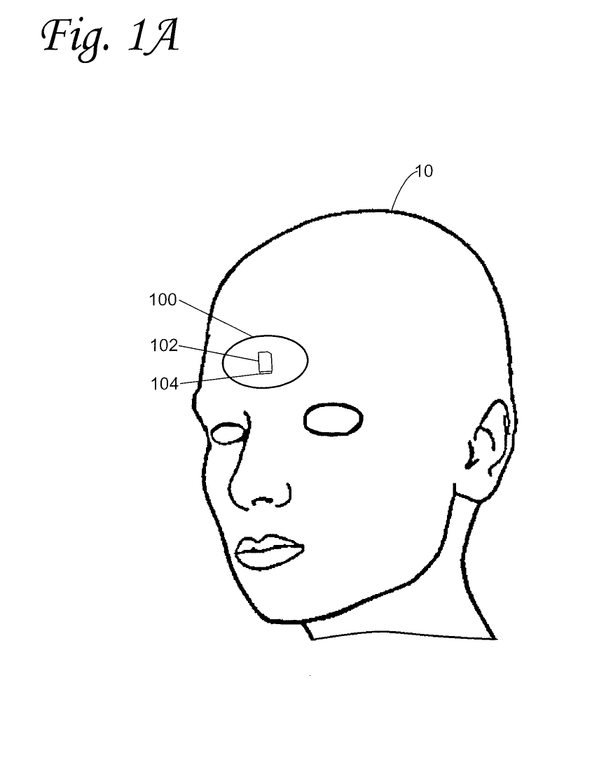

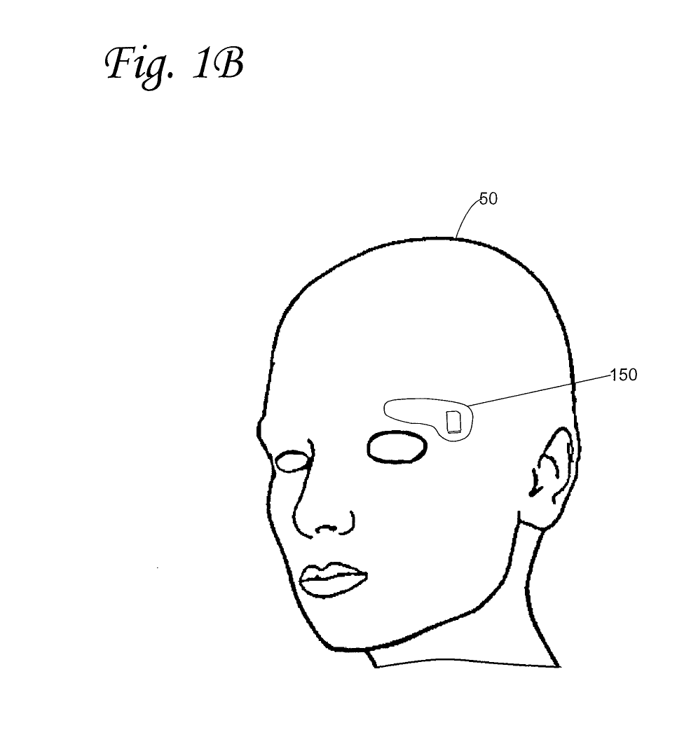

A subject 10 is depicted in FIG. 1A wearing a monitoring patch 100 according to the present invention. The monitoring patch 100 may be a flexible substrate including an adhesive for attaching the monitoring patch 100 to, e.g., a subject's forehead. Non-stick, protective material may cover the adhesive of the monitoring patch 100 prior to use such that it must be removed before attaching the monitoring patch 100 to the subject 10.

The monitoring patch 100 may be attached (e.g., adhered, etc.) to selected areas of the subject's head. For example, the monitoring patch 100 may be attached to the subject's forehead and temple such that it is overlying the frontal and temporal lobes in order to monitor EEG and EOG activity and the temporalis muscle in order to monitor EMG activity, etc.

The monitoring patch 100 depicted in FIG. 1A may include a memory device slot 102 and a memory device 104. The memory device 104 may be any non-volatile storage device. It may be preferred that the memory device 104 be in the form of a flash memory device, such as, e.g., Compact Flash (CF), MultiMedia Card (MMC), Secure Digital (SD), Memory Stick, xD, RS-MMC, miniSD, microSD, Intelligent Stick, etc. In other embodiments, the monitoring patch may include a non-removable storage device. Further, in at least another embodiment, the monitoring patch may include a volatile storage device.

The memory device slot 102 may be a slot designed to receive the memory device 104. When the memory device 104 is inserted into the memory device slot 102 (as shown), the electrical contacts of the memory device 104 may contact the electrical contacts of the memory device slot 102 to allow communication between the memory device and other components on the patch 100. In at least one embodiment, the memory device 104 may fit within the memory device slot 102 with an interference fit. Further, in at least another embodiment, the slot 102 may include a latch or another retention device for securing the memory device 104 within the slot 102.

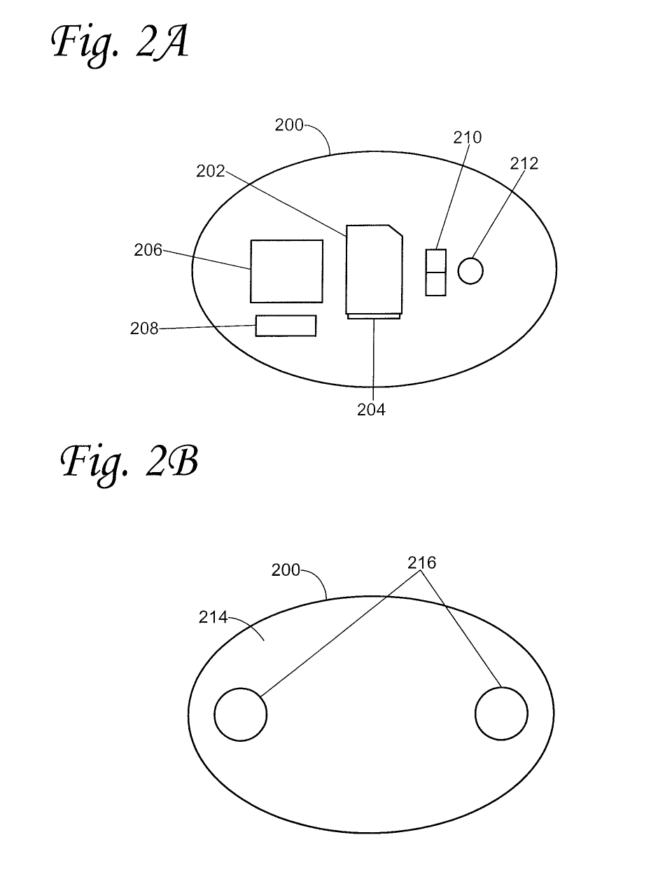

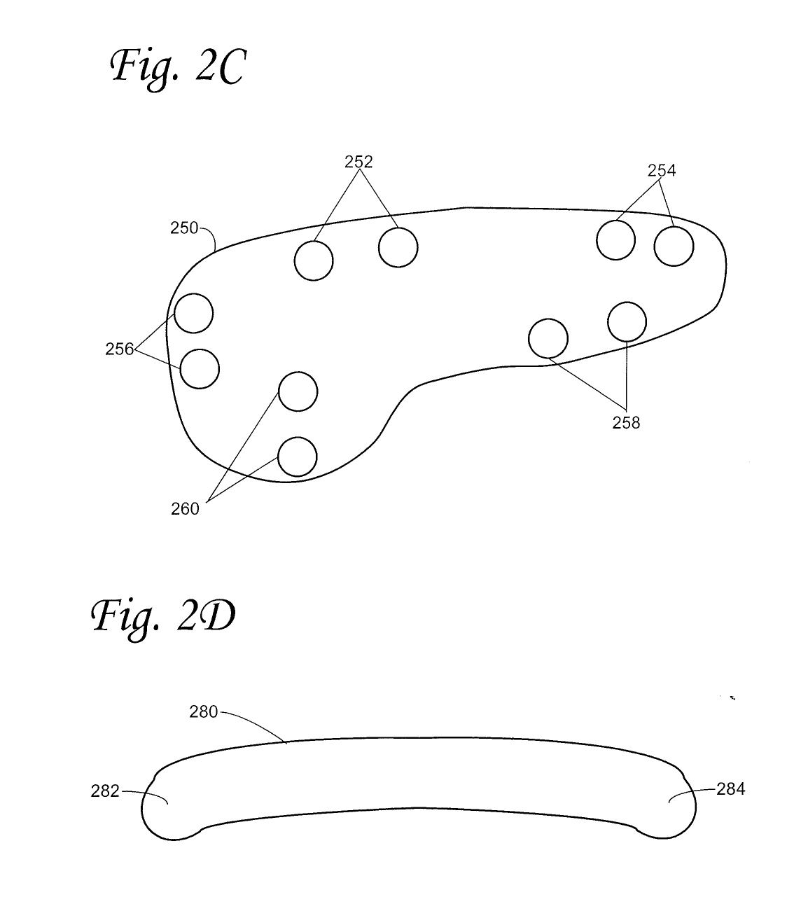

A subject 50 is depicted in FIG. 1B wearing a monitoring patch 150 according to the present invention. The monitoring patch 150 may be similar to the monitoring patch 100 described herein within reference to FIG. 1A. However, the monitoring patch 150 may be shaped to curve around the eye of the subject 50 such that the patch may be positioned about the subject's temporalis muscle, ocular cavity, etc. In other embodiments according to the invention, the monitoring patch may be sized such that may extend across a subject's forehead from the left temporalis muscle to the right temporalis muscle (see, e.g., FIG. 2D).

Another embodiment of a monitoring patch 200 according to the present invention is depicted in FIG. 2A. The monitoring patch 200 may include a memory device slot 202, a memory device 204, electronic apparatus 206, power source 208, power switch 210, and indicator light 212.

The memory device slot 202 and the memory device 204 may be substantially similar to the memory device slot 102 and the memory device 104 as described with reference to FIG. 1. The memory device slot 202 may be electrically coupled to the electronic apparatus 206 such that data from the electronic apparatus 206 may be stored on the memory device 204. As described herein, “electrically coupled” may be any electrical connection, e.g., using a conductive material such as wire connection, flexible circuit board, printed circuit board, etc.

The electronic apparatus 206 may include a microcontroller, microprocessors, EEG apparatus, power management units, analog-to-digital converters, digital signal processors, input/output (I/O) ports, etc. The electronic apparatus 206 may include an I/O port that is electrically coupled to the memory device slot 202. Such electrical coupling may be in the form of any suitable interface, e.g., serial data connection, parallel data connection, Advanced Technology Attachment (ATA), Small Computer System Interface (SCSI), Serial Advanced Technology Attachment (SATA), Universal Serial Bus, IEEE 1394, etc.

The EEG apparatus may include microcontrollers, microprocessors, power management units, analog-to-digital converters, digital signal processors, I/O ports, etc. The EEG apparatus may be integrated with the electronic apparatus on a single microchip. In other embodiments, the EEG apparatus may include multiple electronic components electrically coupled to each other on, e.g., a printed circuit board. In at least one embodiment, the electronic apparatus including EEG apparatus may be attached to a flexible circuit board. The I/O ports of the EEG apparatus may be electrically coupled to one or more electrodes. The one or more electrodes may transmit an electrical signal. The electrical signal may be an analog signal which may be converted to digital data with an analog-to-digital converter. The digital data representative of the analog signal may be stored on the memory device 204. In other embodiments, an analog signal from the one or more electrodes may be stored on a storage apparatus, e.g., magnetic tape, etc.

The monitoring patches according to the present invention may be described as being “self-contained.” As used herein, a self-contained monitoring patch may be defined as having all parts necessary for its operation (e.g., EEG apparatus, electronic apparatus, etc.) located within or on the patch itself (e.g., the substrate of the patch) such that no parts are located a distance away from the patch (e.g., no parts are located a distance away from the patch and connected via a dangling wire or other connection).

The EEG apparatus may continuously sample the neural activity of the subject at any suitable frequency, e.g., about 140 hertz or less, about 100 hertz or less, etc. to monitor the neural activity of the subject that oscillates at about 0.5 hertz or more, about 2 hertz or more, about 70 hertz or less, about 90 hertz or less, etc. In at least one embodiment, the EEG apparatus may monitor neural activity of the subject that oscillates between about 0.5 hertz to about 70 hertz. Further, the rate at which the EEG apparatus may sample the neural activity of the subject may be selectable by, e.g., a switch or an administrator prior to attaching the monitoring patch to the subject. In other embodiments, the EEG apparatus of a monitoring patch may be designed to sample at a selected sampling rate and the monitoring patch may be selected for a particular subject based what sampling rate the EEG apparatus of the monitoring patch may be designed to sample.

Also, the EEG apparatus may acquire and/or store data relating to the neural activity of the subject at selected intervals, e.g., 1 minute of every 5 minutes, 15 seconds of every 1 minute, etc. The selected interval during which data is stored may be chosen in view of the amount of data capable of being stored within the system.

The monitoring patch 200 may monitor the neural activity of the subject for a selected period of time. As used herein, “monitor” or “monitoring” may be defined as any activity that includes acquiring signal activity. As such, “monitor” or “monitoring” may include recording signal activity, analyzing signal activity, numerically transforming signal activity, providing feedback in response to signal activity, etc.

The selected period of time (that the monitoring patch may monitor the neural activity of the subject) may be hours, days, weeks, or even months. Typically, the selected period of time may be a time period correlated to a specific task of a subject. For example, a truck driver may drive a route from Minneapolis, Minn. to Columbus, Ohio. The truck driver may place the patch on his forehead before leaving Minneapolis and may remove the patch upon arriving in Columbus. In this scenario, the selected time period would be the length of time it takes for the truck driver to drive from Minneapolis to Columbus, which may vary depending on traffic conditions, vehicle problems, etc.

Additionally, the patch may monitor the state of vigilance of the driver while driving (e.g., to evaluate for periods of microsleeps) and during non-driving periods to, e.g., measure the sleep duration and sleep cycles during such non-driving periods. Such analysis may be utilized by a trucking company, governmental agency (e.g., the National Transportation Safety Board), etc. to verify that occupational guidelines regarding sleep duration are being achieved. Also, for example, a subject may need to be tested for generalized or partial seizures (e.g., focal motor or complex partial seizures). The monitoring patch may monitor the epileptiform activity of a subject during a period of time (e.g., 1-30 days or more).

The electronic apparatus 206 and/or other components included in the monitoring patch 200 may be powered by a power source 208. The power source 208 may be, e.g., a watch battery or a fuel cell. The power source 208 may be removable or non-removable, rechargeable or non-rechargeable, etc. The power source 208 may be electrically coupled to the electronic apparatus 206 and/or any other device of the monitoring patch 200 that may need power.

The monitoring patch 200 may be turned “on” using power switch 210. The power switch 210 may be any kind of two or more position switch. The power switch 210 may be electrically coupled to the electronic apparatus 206 and/or power supply 208. The power switch 210 may have two positions: “on” and “off” When the power switch 210 is in the “on” position, the monitoring patch 200 may monitor the EEG signals from the electrodes 216 (described below with reference to FIG. 2B) and record such signals to the memory device 204. When the power switch 210 is in the “off” position, the monitoring patch 200 may be dormant, i.e., not monitoring the EEG. The power switch 210 may have more than two positions for different modes of operation of the monitoring patch 200. For example, the power switch 210 may have a position for a “download” mode in which data may be removed from the monitoring patch 200. In other embodiments, the monitoring patch could be turned “on” by attaching the patch a subject's forehead (e.g., the patch may include electrodes capable of sensing when the patch is contacting skin—at which time apparatus on the patch may be turned “on”).

The monitoring patch 200 may further include an indicator light 212. The indicator light 212 may be a single LED (as depicted). In at least one embodiment, however, the indicator light 212 may consist of one or more LEDs, OLEDs, and/or LCDs. The indicator light 212 may indicate to the user the mode or state of the monitoring patch 200. For example, if the indicator light is “on,” then the indicator light 212 may be indicating that the monitoring patch 200 is monitoring EEG signals from the electrode and storing such signals on the memory device 204. Also, for example, the indicator light 212 may “blink” to indicate the power source 208 is running low on power.

Another view of monitoring patch 200 according to the present invention is depicted in FIG. 2B. The side of the monitoring patch 200 depicted in FIG. 2B is the side that may be attached to a subject as shown in FIG. 1 and it may include an adhesive 214 and electrodes 216.

In the embodiment depicted in FIG. 2B, the monitoring patch 200 includes two electrodes 216. One of the two electrodes may be a reference electrode. In other embodiments, however, the monitoring patch may include more than two electrodes. The electrodes 216 may be electrically coupled to the electronic apparatus 206, or more specifically, an I/O port of the EEG apparatus of the electronic apparatus 206. For example, a wire, circuit trace, etc. may extend between an electrode and a signal interface of the EEG apparatus.

The electrodes 216 may be located on the monitoring patch 200 to correspond to specific areas of the subject's brain when the monitoring patch 200 is attached to the head of a subject (e.g., see monitoring patch 100 on subject 10 depicted in FIG. 1). For example, in the embodiment depicted in FIG. 2B, the electrodes 216 are located to correspond to the frontal lobes of the subject's brain. In other embodiments, the electrodes 216 may be located on the monitoring patch 200 to correspond to the subject's eye movements, frontalis muscle, temporalis muscle, temporal lobes, temple, forehead, etc.

The monitoring patch 200 may include adhesive 214 for attaching the monitoring patch 200 to, e.g., a subject's forehead as shown in FIG. 1. The adhesive 214 may preferably be any skin-compatible, pressure-sensitive adhesive that may adhere to a subject and that may be removed without significantly damaging the subject's skin. Further, the adhesive 214 and/or the monitoring patch 200 may include apertures such that the patch 200 is “breathable.” Also, the adhesive 214 and/or monitoring patch 200 may be flexible so that it may conform to uneven surfaces, such as a subject's forehead. The adhesive 214 may cover, partially cover, or not cover the electrodes 216. In at least one embodiment, the adhesive 214 may be thinner over the electrodes 216 than the remainder of the monitoring patch 200 such that sufficient conductivity can be obtained between the electrodes and the subject's skin. In at least one embodiment, the adhesive may be in the form of an adhesive pad or cushion. A non-stick, protective backing material may be located over the adhesive of the monitoring patch that may be peeled-off before attaching the monitoring patch to a subject. The monitoring patch may be able to sense when the backing material is removed from patch and thereby turn “on” the patch.

The monitoring patch 200 depicted in FIGS. 2A & 2B is oval shaped. The monitoring patch, however, may be any shape. For example, the monitoring patch may be a specific shape to position the electrodes over selected areas of the subject's head. For instance, the monitoring patch may be curved (e.g., like a banana or crescent) such that the electrodes may simultaneously monitor EEG, EOG, and EMG activity. Further, for example, the monitoring patch may be a specific shape that enhances its ability to maintain adequate adherence to the subject. Also, the monitoring patch 200 may be provided in a variety of different sizes to, e.g., correspond to subjects having different-sized heads.

The monitoring patch according to the present invention may also include EMG apparatus to record the muscular activity of electrical potential across muscular membranes and EOG apparatus to record the ocular activity of a subject. The EMG apparatus and/or EOG apparatus may be electrically coupled to one or more electrodes in the same manner as described with the EEG apparatus.

The monitoring patch 250 depicted in FIG. 2C may be similar to the monitoring patch 200 depicted in FIGS. 2A & 2B except that the monitoring patch 250 may include additional electrodes and/or electronic apparatus and may be shaped such the electrodes are located near selected portions of the subject's head. The monitoring patch 250 includes EEG electrodes 252 located to correspond to a subject's temporal lobes, EEG electrodes 254 located to correspond to a subject's frontal lobes, EMG electrodes 256 located to correspond to a subject's temporalis muscle, EOG electrodes 258 located to correspond to a subject's ocular cavity, and reference electrodes 260. Further, the monitoring patch 250 may include EEG, EOG, and EMG apparatus (although not depicted) to monitor and collect data from the electrodes. FIG. 2C is a rear view of monitoring patch 250, and therefore, depicts the side of the monitoring patch 250 that will be adhered to the subject. One example of a monitoring patch that is similar to monitoring patch 250 is shown adhered to subject 50 in FIG. 1B.

Although multiple electrodes are depicted for each type or class, only one electrode may be required at each location. The use of multiple electrodes provides redundancy (if, e.g., an electrode loses contact, an electrode malfunctions, etc.). Also, any of the electrodes on the patch may be used as a reference electrode (if, e.g., the use of a different electrode provides a better reference signal, a reference electrode malfunctions, etc.).

The monitoring patch 280 depicted in FIG. 2D may be similar to the monitoring patches 200, 250 depicted in FIGS. 2A-2C except that the monitoring patch 280 may sized and shaped to extend across the forehead of a subject from a first end 282 to a second end 284. Each end 282, 284 may correspond to a subject's temporalis muscle. The patch 280 may include multiple electrodes located throughout the patch 280 to monitor any portion of the subject's head.

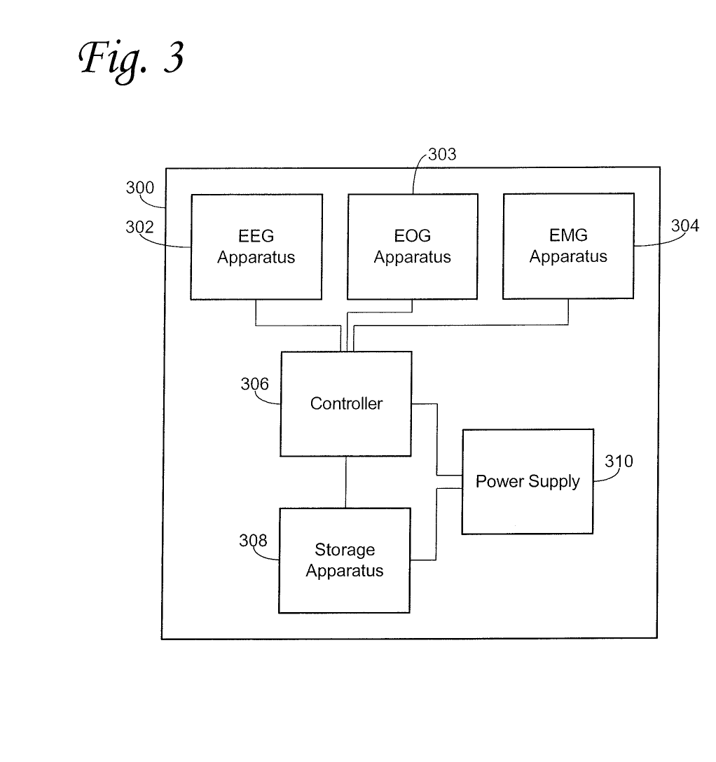

FIG. 3 is a diagrammatic representation of one exemplary embodiment of a monitoring patch 300 according to the present invention. The monitoring patch 300 may include EEG apparatus 302, EOG apparatus 303, EMG apparatus 304, a controller 306, storage apparatus 308, and a power supply 310.

The EEG apparatus 302 may include microcontrollers, microprocessors, analog-to-digital converters, digital signal processors, I/O ports, etc. The EEG apparatus 302 may be capable of recording the neural activity of electrical potential across cell membranes. The changes in electrical potential in the cortex contain rhythmical activity, which typically occur at frequencies of about 0.5 hertz to about 70 hertz. The EEG apparatus 302 may continuously sample the neural activity of the subject at about 100 hertz or less, 60 hertz or less, etc. and may monitor the neural activity of the subject that oscillates between about 0.5 hertz or more, about 70 hertz or less, etc. In at least one embodiment, the EEG apparatus 302 may monitor the neural activity of the subject that oscillates between about 0.5 hertz to about 70 hertz. Also, the EEG apparatus may monitor the neural activity of the subject at selected intervals, e.g., 1 minute for every 5 minutes or 15 seconds for every 1 minute.

The EOG apparatus 303 may include microcontrollers, microprocessors, analog-to-digital converters, digital signal processors, I/O ports, etc. The EOG apparatus 303 may be capable of recording the ocular activity of a subject. Changes in electrical potential near the subject's eye as a result of ocular activity may oscillate between about 0.5 hertz and about 200 hertz. The EOG apparatus 303 may sample the ocular activity of the subject at about 180 hertz or less, about 100 hertz or less, etc., and may monitor the ocular activity of the subject that oscillates at about 0.5 hertz or more, about 2 hertz or more, about 100 hertz or less, about 200 hertz or less, etc. Further, the rate at which the EOG apparatus may sample the ocular activity of the subject may be selectable by, e.g., a switch or an administrator prior to attaching the monitoring patch. In other embodiments, the EOG apparatus of a monitoring patch may be designed to sample at a selected sampling rate and the monitoring patch may be selected for a particular subject based what sampling rate the EOG apparatus of the monitoring patch may be designed to sample. EOG monitoring may be limited to periods when the EEG apparatus and/or EMG apparatus indicate that a subject is sleeping.

The EMG apparatus 304 may include microcontrollers, microprocessors, analog-to-digital converters, digital signal processors, I/O ports, etc. The EMG apparatus 304 may be capable of recording the muscular activity of electrical potential across muscular membranes. Changes in electrical potential in muscular membranes may oscillate between about 10 hertz or more, about 90 hertz or less, etc. depending on the size of the muscle, the type of muscle, etc. The EMG apparatus 304 may sample the muscular activity of the subject at about 180 hertz or less, about 100 hertz or less, etc., and may monitor the muscular activity of the subject that oscillates at about 0.5 hertz or more, about 10 hertz or more, about 90 hertz or less, about 180 hertz or less, etc. Further, the rate at which the EMG apparatus may sample the muscular activity of the subject may be selectable by, e.g., a switch or an administrator prior to attaching the monitoring patch. In other embodiments, the EMG apparatus of a monitoring patch may be designed to sample at a selected sampling rate and the monitoring patch may be selected for a particular subject based what sampling rate the EMG apparatus of the monitoring patch may be designed to sample. In at least one embodiment, the EMG apparatus 304 may monitor the muscular activity of the temporalis muscle and/or frontalis muscle of a subject that corresponds to REM sleep, e.g., the muscular activity of the temporalis muscle and/or frontalis muscle that oscillates at about 10 hertz or more, about 90 hertz or less, etc. Further, the EMG apparatus 304 may measure electrical potential at about 25 microvolts or more, about 50 millivolts or less, etc.

As described above, the monitoring patch according to the current invention may take any suitable shape. A monitoring patch including EMG apparatus may be specifically shaped to locate the one or more electrodes of the EMG apparatus over the temporalis and/or frontalis muscles to monitor for REM sleep and/or the one or more electrodes of the EOG apparatus proximate the subject's eyes to monitor ocular activity.

Although the embodiment of the monitoring patch 300 depicted in FIG. 3 includes EEG apparatus, EOG apparatus, and EMG apparatus, monitoring patches according to the present invention may include different combinations of the less than the three apparatuses, e.g., EEG apparatus and EOG apparatus, or EEG apparatus and EMG apparatus.

The EEG apparatus 302, the EOG apparatus 303, and the EMG apparatus 304 may include one or more electrodes similar to the electrodes 216 described herein with reference to FIG. 2B. The electrodes may be integral to the EEG apparatus 302, the EOG apparatus 303, and/or EMG apparatus 304. In other embodiments, the electrodes may by spaced away from the EEG apparatus 302, the EOG apparatus 303, and/or the EMG apparatus 304 and attached to the EEG apparatus 302, the EOG apparatus 303, and/or EMG apparatus 304 by any suitable connection, e.g., a wire, printed circuit board, etc. Still, in other embodiments, the EEG apparatus 302, the EOG apparatus 303, and the EMG apparatus 304 may share and utilize the same electrode(s).

The controller 306 may control that operation of the monitoring patch 300. For example, the controller 306 may control the EEG, EOG, and EMG signal recording operations taking place with the EEG apparatus 302, the EOG apparatus 303, and the EMG apparatus 304. The controller 306 may receive such EEG, EOG, and EMG data, may process such data, and then store the processed data on the storage apparatus 308. The controller 306 may be any standard microcontroller and/or microprocessor (e.g., a PIC microcontroller). The controller 306 may include one or more central processing unit, I/O ports (e.g., serial ports, USB ports), volatile memory, nonvolatile memory, clock generators, analog-to-digital converters, etc.

The storage apparatus 308 may be any volatile or non-volatile electronic storage device that is capable of storing data from the controller 306. In at least one embodiment, the storage apparatus 308 may include removable non-volatile memory such as the memory device 104 described herein with reference to FIG. 1.

The power supply 310 may be similar to the power source 208 described herein with respect to FIG. 2A. The power supply 310 may be electrically coupled to the storage apparatus 308 and the controller 306 to provide power to the storage apparatus 308 and the controller 306. In other embodiments, the power supply 310 may also be electrically coupled to the EEG apparatus 302, the EOG apparatus 303, and/or the EMG apparatus 304.

The controller 306 may be further electrically coupled to the storage apparatus 308 for transferring data between the storage apparatus 308 and the controller 306. The connection between the controller 306 and the storage apparatus 308 may be a data transmission connection that may utilize any suitable data transmission protocol. Examples of some potentially suitable data transmission protocols may include serial data connection, parallel data connection, Advanced Technology Attachment (ATA), Small Computer System Interface (SCSI), Serial Advanced Technology Attachment (SATA), Universal Serial Bus, IEEE 1394, etc.

The controller 306 may be still further electrically coupled to the each of the EEG apparatus 302, the EOG apparatus 303, and the EMG apparatus 304. The EEG apparatus 302, the EOG apparatus 303, and/or the EMG apparatus 304 may transmit analog and/or digital data to one or more I/O ports of the controller 306. In at least one embodiment, the EEG apparatus 302, the EOG apparatus 303, and/or the EMG apparatus 304 may transmit raw analog electrical signals to the controller 306, which may convert the raw signals into a digitized form. The EEG apparatus 302, the EOG apparatus 303, and/or the EMG apparatus 304 may include any electronic components that facilitate the recording and/or detection of the electromagnetic and/or electrical activity indicative of neural activity, ocular activity, and/or muscular activity of a subject. For example, the EEG apparatus 302, EOG apparatus 303, and/or the EMG apparatus 304 may include capacitive electronic components (e.g., capacitors) to filter electronic noise below or above selected frequency thresholds.

Although the different components (i.e., EEG apparatus 302, EOG apparatus 303, EMG apparatus 304, controller 306, storage apparatus 308, power supply 310) of monitoring patch 300 are shown in FIG. 3 as being separate, such components may be completely integrated or partially integrated into one or more units. For example, such components may reside in one single microelectronic chip, may be located on the same flexible circuit board, etc.

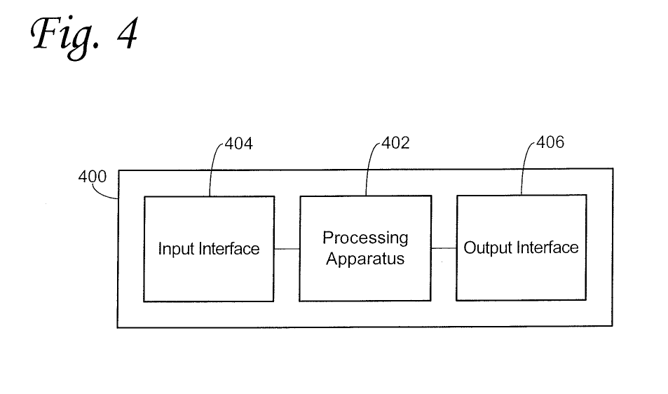

FIG. 4 is a diagrammatic representation of one exemplary embodiment of an analysis system 400 according to the present invention. The analysis 400 may be utilized by a user to analyze the EEG, EOG, and/or EMG data recorded from, e.g., the EEG apparatus 302, the EOG apparatus 303, and/or the EMG apparatus 304 of the monitoring patch 300 shown in FIG. 3.

The analysis system 400 may include a processing apparatus 402, an input interface 404, an output interface 406, and a power supply 408. The analysis system 400 may be a personal computer running an operating system such as Microsoft Windows, GNU/Linux, Apple OS X, etc. In other embodiments, the analysis system 400 may be a personal data assistant (PDA), a laptop computer, a cellular telephone, an ultra-mobile personal computer (UMPC), etc.

The input interface 404 may be an interface designed to receive the data recorded using the EEG apparatus, the EOG apparatus 303, and/or the EMG apparatus of a monitoring patch. The input interface 404 may include a slot for receiving a removable memory device such as the memory device 204 of FIG. 2A. In other embodiments, the input interface 404 may be an I/O port such as a serial data port, a parallel, data port, a USB data port, etc. that may be connectable to the storage device of the monitoring patch. In these embodiments, a data transmission cable (e.g., a USB cable) may be connected to the input interface 404 of the analysis system 400 and to the monitoring patch to download the data from the monitoring patch to the analysis system 400.

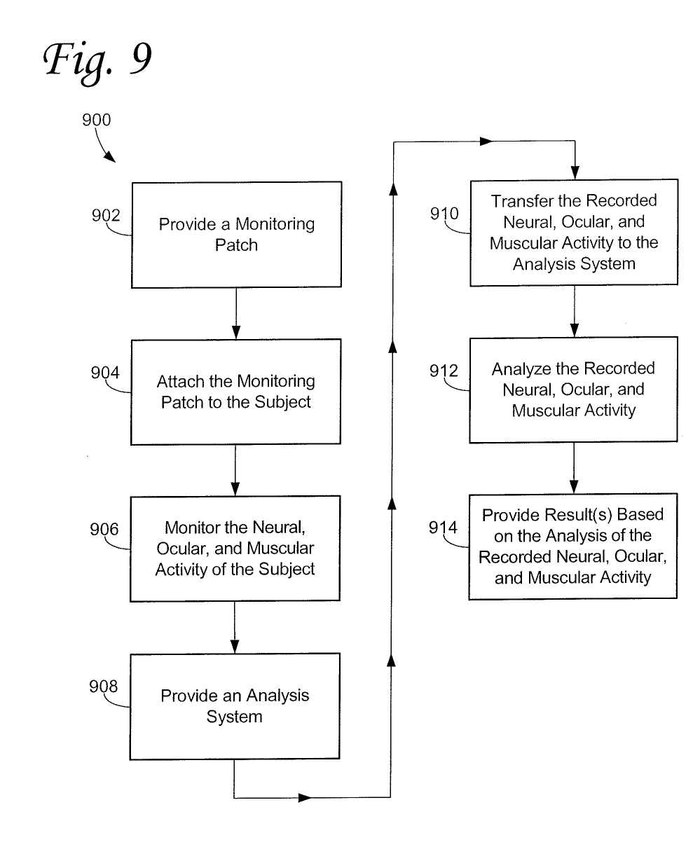

The output interface 406 may be an interface for displaying the EEG, EOG, and/or EMG data and an analysis of such data to the user of the analysis system 400. For example, the user may be a trucking company administrator. Upon returning from a job, a truck driver may submit the monitoring patch (or just the memory device of the monitoring patch) worn by the driver to the administrator. The administrator may connect the monitoring patch into the input interface of the analysis system. The analysis system 400 may, either autonomously or under control of the administrator, download the EEG, EOG, and/or EMG data from the monitoring patch (or memory device) and analyze that data to determine, e.g., how many hours the truck driver had slept during the monitoring period, how vigilant the trucker driver had been during the monitoring period, and/or how many hours the truck drive had slept during the during the rest periods. The output interface 406 of the analysis system 400 may display such determined results on a monitor (e.g., a CRT, a LCD, etc.), may print such determined results on a printer (e.g., see FIG. 9), may record the results (and/or data) in another medium, etc.

In another embodiment, the user of the analysis system 400 may be a doctor. A doctor may use the analysis system to determine if a subject may have a disorder of hypersomnia, insomnia, or a circadian rhythm disorder, etc. In this example, the monitoring patch and analysis system may operate as a seizure screening device.

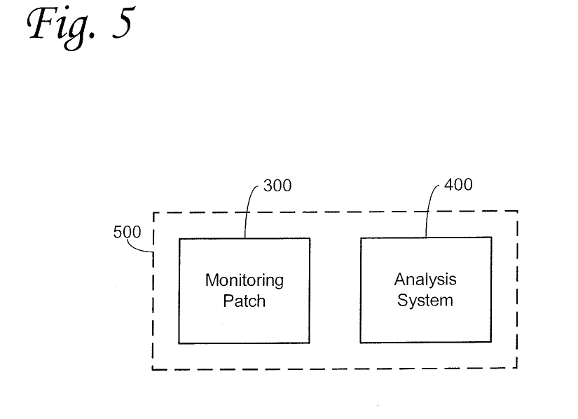

FIG. 5 is a diagrammatic representation of one exemplary embodiment of a monitoring and analysis system 500 according to the present invention. The system 500 may include the monitoring patch 300 of FIG. 3 and the analysis system 400 of FIG. 4.

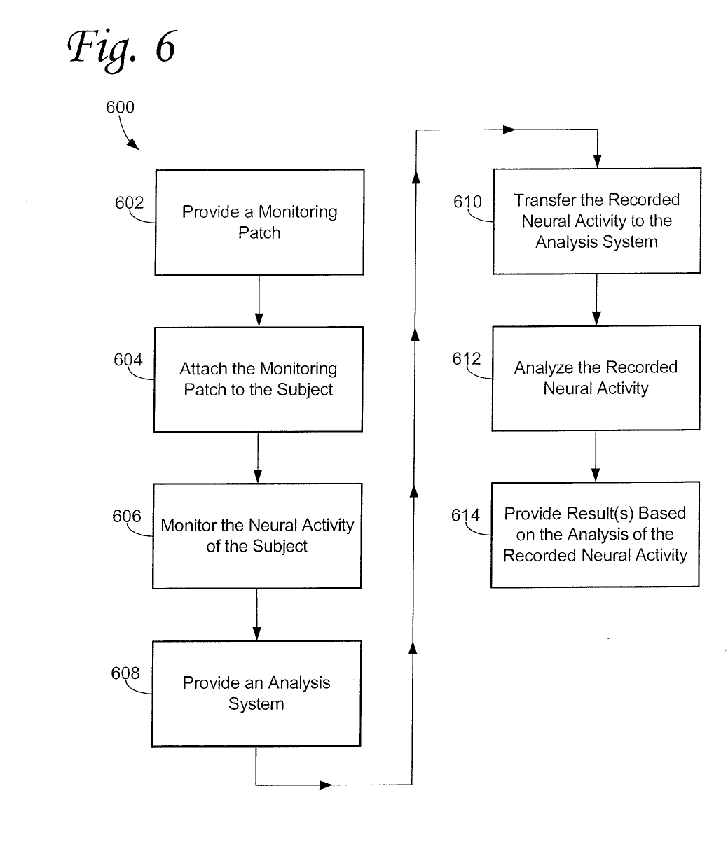

FIG. 6 depicts one exemplary method 600 of monitoring a subject's neural activity according to the present invention. The method 600 includes providing a monitoring patch 602 and adhering the monitoring patch to the subject 604. The monitoring patch may be similar to the monitoring patch 200 described herein with reference to FIGS. 2A & 2B. Attaching the monitoring patch to the subject 604 may include peeling a protective layer from the adhesive surface of the monitoring patch, locating the patch proximate to the portion of the subject's brain to be monitored, and applying the patch to the portion of the subject's head (e.g., forehead or temple). In other embodiments, step 604 may further include applying an adhesive substance to the rear side of a monitoring patch before applying the patch to the subject.

After attaching the monitoring patch 604, method 600 further includes monitoring the neural activity of the subject 606. Monitoring the neural activity of the subject 606 may include the use of the monitoring patch to record EEG signals from the subject's head. The EEG signals may be recorded to, e.g., a memory device.

The method 600 further includes providing an analysis system 608. The analysis system provided in step 608 may be the analysis system 400 described herein with reference to FIG. 4. After the neural activity (i.e., the EEG signals) has been monitored 606, the method further includes transferring the recorded neural activity data to the analysis system 610. Transferring the recorded neural activity data to the analysis system 610 may include utilizing a data transfer cable between the monitoring patch and analysis system, removing a memory device from the monitoring patch and connecting it to the analysis system, wirelessly transmitting the recorded neural activity data, etc.

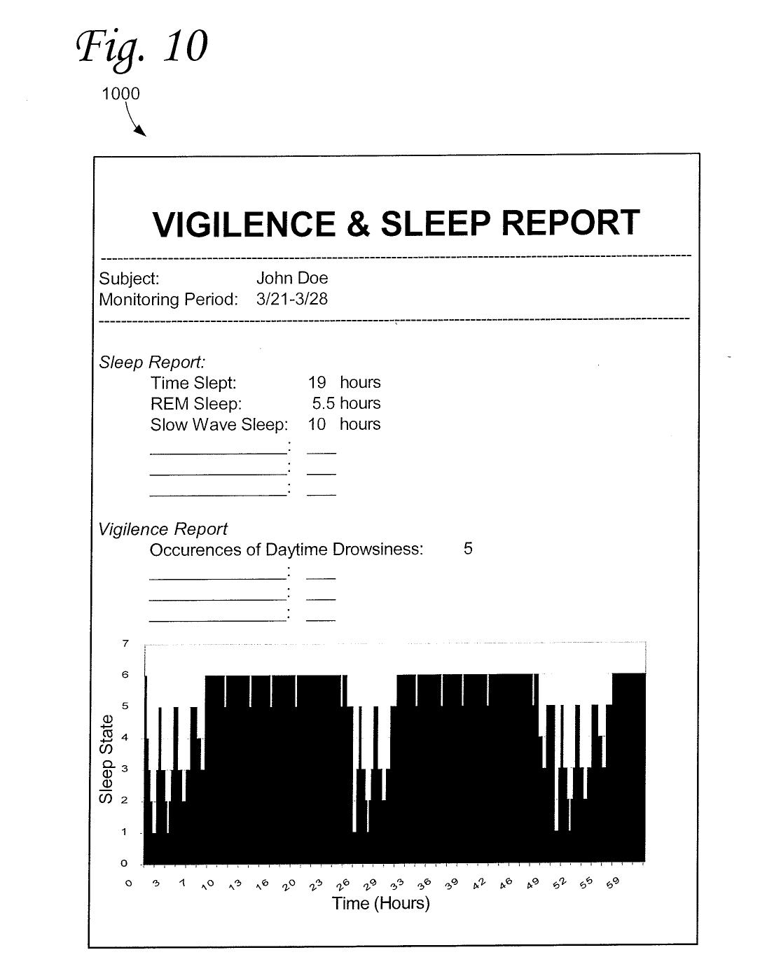

After at least a portion of the recorded neural activity data is transferred to the analysis system, the method 600 may include analyzing the recorded neural activity data 612 with the analysis system. The analysis 612 may include any suitable analog and/or digital signal analysis. The analysis 612 may, either autonomously or under control of the administrator, analyze that data to determine, e.g., how many hours the subject had slept during the monitoring period, how vigilant the subject had been during the monitoring period, how many hours subject slept between driving periods, the pattern of sleep periods that could lead to the diagnosis of a sleep disorder, etc. After at least a portion of the neural activity data is analyzed 612, the method 600 may include providing result(s) based on the analysis of the recorded neural activity data 614. Such results may be provided on an electronic display (e.g., a CRT, a LCD, etc.), may be printed on, e.g., a piece of paper, may be recorded in another storage medium (e.g., a CD-ROM). For example, FIG. 10 depicts one exemplary output report 1000 that may be provided by the methods and/or systems according to the present invention. The output report 1000 may be printed on a piece of paper and may include subject name, monitoring period, number of hours of sleep, number of hours of REM sleep, number of hours of slow wave sleep, occurrences of daytime drowsiness, time-lapsed EEG graph, time-lapsed EOG graph, time-lapsed EMG graph, hypnograms of various states of sleep and wakefulness, etc. In other embodiments, the results may be stored as a digital file (e.g., binary file) on the analysis system (e.g., a digital file within a database).

The analysis system may store the results for many different subjects, many different time periods, etc. As such, the analysis system may store such recorded data for multiple subjects.

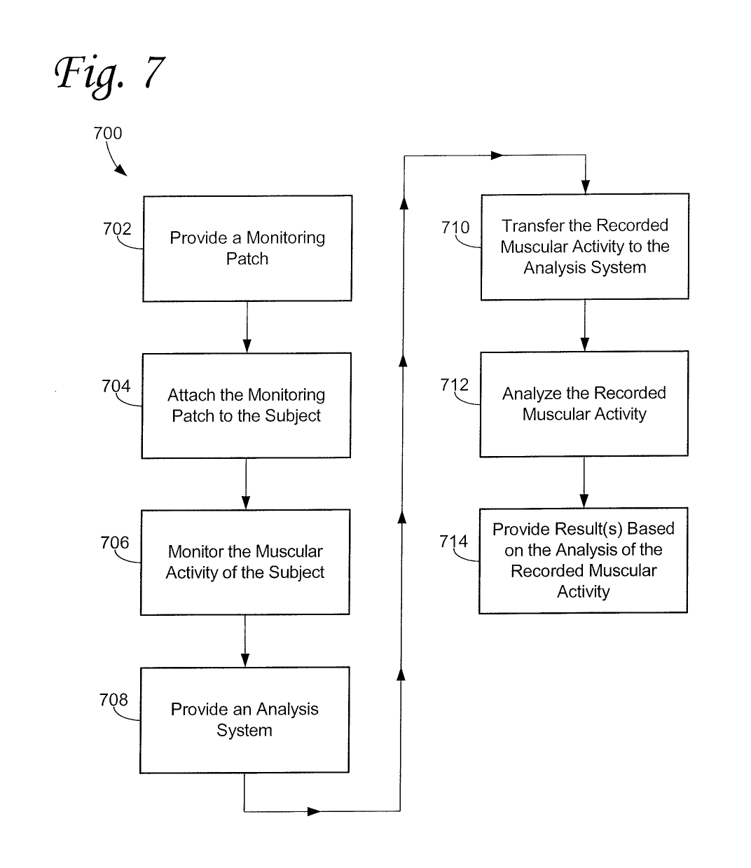

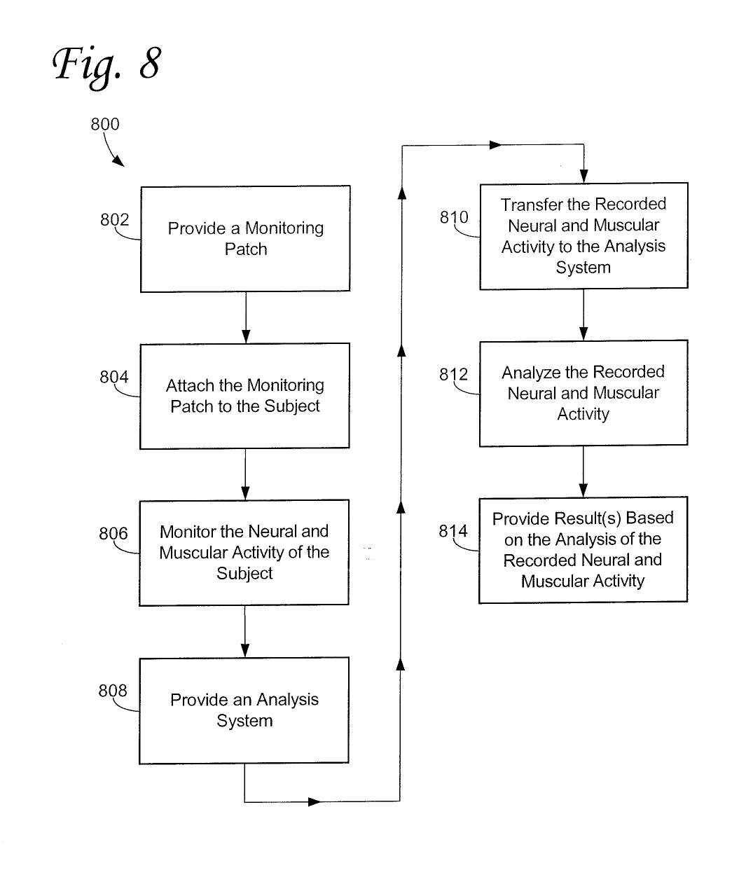

FIG. 7 depicts an exemplary method of monitoring a subject's muscular activity 700 according to the present invention, FIG. 8 depicts an exemplary method of monitoring a subject's neural and muscular activity 800 according to the present invention, and FIG. 9 depicts an exemplary method of monitoring a subject's neural, muscular, and ocular activity 900 according to the present invention. The methods 700, 800, and 900 are similar method 600 except that method 700 involves monitoring and analysis of muscular activity using, e.g., EMG, method 800 involves monitoring and analysis of both neural and muscular activity using, e.g., EEG and EMG, and method 900 involves monitoring and analysis of neural, ocular, and muscular activity using, e.g., EEG, EOG, and EMG. The data generated may be analyzed, used, stored, etc. as described with the other systems described herein.

The systems, methods, devices according to the present invention may further include verification/integrity apparatus and methods. Such verification/integrity apparatus and methods may be utilized to determine the identity of the subject wearing the patch during the monitoring period, to determine if the patch is worn continuously by the subject during the monitoring period, to determine if the memory device of the monitoring patch is the same memory device as used throughout the monitoring period, etc. The verification/integrity apparatus may include a unique digital signature (and/or fingerprint) corresponding to each memory device and/or each monitoring patch. The results from the verification/integrity apparatus and methods may be provided along with the results of method 600 as described herein (e.g., on an electronic display, a paper output report, etc.). In some embodiments, the verification/integrity apparatus and methods may involve monitoring electrodes on the patch to determine if the patch was removed from the subject during the monitoring period. For example, if continuity between the electrodes is interrupted in a manner that could indicate removal of the patch from the subject, the data may be flagged as potentially suspect or bad because the loss of continuity may indicate that the patch was removed from the subject.

Further, the verification/integrity apparatus and methods may analyze a subject's past data to determine a unique EEG, EOG, EMG, etc. “signature” or “fingerprint” of that particular subject. That unique “signature” or “fingerprint” can be compared to the data collected during a monitoring period to verify that the particular subject was wearing the monitoring patch during such monitoring period. The verification/integrity apparatus and methods described herein may be utilized to, e.g., prevent a subject from “faking” sleep activity, vigilance activity, etc. during a monitoring period by removing the monitoring patch and attaching it to another subject, a mechanical device, an electronic device, an animal, etc.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A system for monitoring the neural, muscular, and ocular activity of a subject, the system comprising:
a self-contained monitoring patch, wherein the monitoring patch comprises:
electroencephalography apparatus to monitor neural activity of the subject during a selected time period, wherein the electroencephalography apparatus comprises an electrode;
electromyography apparatus to monitor muscular activity of the subject during the selected time period, wherein the electromyography apparatus comprises an electrode;
electrooculography apparatus to monitor ocular activity of the subject during a selected period of time, wherein the electrooculography apparatus comprises an electrode;
storage apparatus electrically coupled to the electroencephalography apparatus to store neural activity data, to the electromyography apparatus to store muscular activity data, and to the electrooculography apparatus to store ocular activity data;
a power supply; and
adhesive to attach the monitoring patch to the subject; and
an analysis system to analyze the neural activity data, the muscular activity data, and the ocular activity data, wherein the analysis system comprises:
an input interface coupleable to the storage apparatus to receive the neural activity data, the muscular activity data, and the ocular activity data;
processing apparatus coupled to the input interface to analyze the neural activity data, the muscular activity data, and the ocular activity data; and
an output interface coupled to the processing apparatus to output results from the analysis of the neural activity data, the muscular activity data, and the ocular activity data.

2. The system of claim 1, wherein the electroencephalography apparatus of the monitoring patch samples the neural activity of the subject at about 140 hertz or less.

3. The system of claim 1, wherein the electroencephalography apparatus of the monitoring patch monitors the neural activity of the subject that oscillates between about 0.5 hertz to about 70 hertz.

4. The system of claim 1, wherein the electromyography apparatus of the monitoring patch samples the muscular activity of the subject at about 140 hertz or less.

5. The system of claim 1, wherein the electromyography apparatus of the monitoring patch monitors the muscular activity of the subject that oscillates between about 10 hertz to about 90 hertz.

6. The system of claim 1, wherein the electrooculography apparatus of the monitoring patch samples the ocular activity of the subject at about 140 hertz or less.

7. The system of claim 1, wherein the electrooculography apparatus of the monitoring patch monitors the ocular activity of the subject that oscillates between about 10 hertz to about 90 hertz.

8. The system of claim 1, wherein the storage apparatus of the monitoring patch comprises a removable memory device, and wherein the input interface of the analysis system comprises a slot to interface with the removable memory device.

9. The system of claim 1, wherein the monitoring patch further comprises an output interface to transmit the neural activity data, the muscular activity data, and the ocular activity data to the analysis system, wherein the output interface of the monitoring patch is operably coupleable to the input interface of the analysis system.

10. The system of claim 1, wherein the monitoring patch further comprises an indicator to indicate the state of the monitoring patch.

11. The system of claim 1, wherein the processing apparatus of the analysis system analyzes the neural activity data, the muscular activity data, and the ocular activity data from the storage apparatus to determine how long the subject has slept.

12. The system of claim 1, wherein the output interface of the analysis system displays a numerical length of the time the subject has slept.

13. The system of claim 1, wherein the output interface of the analysis system displays a graphical representation of the time the subject has slept.

14. The system of claim 1, wherein the output interface of the analysis system displays a numerical length of the time the subject was vigilant.

15. The system of claim 1, wherein the output interface of the analysis system displays a graphical representation of the vigilance of the subject during the selected time period.

16. The system of claim 1, wherein the processing apparatus of the analysis system analyzes the data from the from the storage apparatus to determine the vigilance of the subject during the selected time period.

17. The system of claim 1, wherein the output interface of the analysis system displays a numerical value representing the vigilance of the subject during the selected time period.

18. The system of claim 1, wherein the output interface of the analysis system displays a numerical value representing the vigilance of the subject during a portion of the selected time period.

19. The system of claim 1, wherein the analysis system further comprises verification apparatus to verify that the monitoring patch was worn continuously by the subject during the selected time period.

20. The system of claim 1, wherein the monitoring patch further comprises verification apparatus to verify that the monitoring patch was worn continuously by the subject during the selected time period.

21. A method for monitoring the neural, muscular, and ocular activity of a subject, the method comprising:
providing a self-contained monitoring patch, wherein the monitoring patch comprises:
electroencephalography apparatus electrically coupled to a storage apparatus;
electromyography apparatus electrically coupled to the storage apparatus;
electrooculography apparatus electrically coupled to the storage apparatus;
one or more electrodes electrically coupled to the electroencephalography apparatus, the electromyography apparatus, and the electrooculography apparatus;
a power supply; and
adhesive;
attaching the monitoring patch to a subject;
monitoring neural activity of the subject with the electroencephalography apparatus during a selected period of time to obtain neural activity data;
monitoring muscular activity of the subject with the electromyography apparatus during a selected period of time to obtain muscular activity data;
monitoring ocular activity of the subject with the electrooculography apparatus during a selected period of time to obtain ocular activity data;
transferring the neural activity data, the muscular activity data, and the ocular activity data from the storage apparatus of the monitoring patch to an analysis system;
analyzing the neural activity data, the muscular activity data, and the ocular activity data using the analysis system; and
outputting a result based on the analysis of the neural activity data, the muscular activity data, and the ocular activity data.

22. The method of claim 21, wherein monitoring the neural activity of the subject with the electroencephalography apparatus during the selected period of time comprises:
sampling the neural activity of the subject;
converting the neural activity to the neural activity data; and
storing the neural activity data on the storage apparatus of the monitoring patch;
wherein monitoring the muscular activity of the subject with the electromyography apparatus during the selected period of time comprises:
sampling the muscular activity of the subject;
converting the muscular activity to the muscular activity data; and
storing the muscular activity data on the storage apparatus of the monitoring patch; and
wherein monitoring the ocular activity of the subject with the electrooculography apparatus during the selected period of time comprises:
sampling the ocular activity of the subject;
converting the ocular activity to the ocular activity data; and
storing the ocular activity data on the storage apparatus of the monitoring patch.

23. The method of claim 22, wherein sampling the neural activity of the subject comprises sampling the neural activity of the subject at about 140 hertz or less.

24. The method of claim 21, wherein monitoring the neural activity of the subject comprises monitoring the neural activity of the subject that oscillates between about 0.5 hertz to about 70 hertz.

25. The method of claim 22, wherein sampling the muscular activity of the subject comprises sampling the muscular activity of the subject at about 140 hertz or less.

26. The method of claim 21, wherein monitoring the muscular activity of the subject comprises monitoring the muscular activity of the subject that oscillates between about 10 hertz to about 90 hertz.

27. The method of claim 22, wherein sampling the ocular activity of the subject comprises sampling the ocular activity of the subject at about 140 hertz or less.

28. The method of claim 21, wherein monitoring the ocular activity of the subject comprises monitoring the ocular activity of the subject that oscillates between about 10 hertz to about 90 hertz.

29. The method of claim 21, wherein the storage apparatus of the monitoring patch comprises a removable memory device, and wherein the input interface of the analysis system comprises a slot to interface with the removable memory device.

30. The method of claim 21, wherein the monitoring patch further comprises an output interface to transmit the neural activity data, the muscular activity data, and the ocular activity data to the analysis system, wherein the output interface of the monitoring patch is operably coupleable to the input interface of the analysis system.

31. The method of claim 30, the method further comprising coupling the output interface of the monitoring patch to the input interface of the analysis system.

32. The method of claim 21, wherein the monitoring patch further comprises an indicator to indicate the state of the monitoring patch.

33. The method of claim 21, wherein analyzing the neural activity data, the muscular activity data, and the ocular activity data comprises determining how long the subject has slept during the selected period of time.

34. The method of claim 21, wherein providing the result through the output interface comprises displaying a numerical length of the time the subject has slept.

35. The method of claim 21, wherein providing the result through the output interface comprises displaying a graphical representation of the time the subject has slept.

36. The method of claim 21, wherein providing the result through the output interface comprises displaying a numerical length of the time the subject was vigilant.

37. The method of claim 21, wherein providing the result through the output interface comprises displaying a graphical representation of the vigilance of the subject during the selected time period.

38. The method of claim 21, wherein analyzing the neural activity data, the muscular activity data, and the ocular activity data comprises determining the vigilance of the subject during the selected time period.

39. The method of claim 21, wherein providing the result through the output interface comprises displaying a numerical value representative of the vigilance of the subject during the selected time period.

40. The method of claim 21, wherein providing the result through the output interface comprises displaying a numerical value representative of the vigilance of the subject during a portion of the selected time period.

41. The method of claim 21, the method further comprising verifying that the monitoring patch was worn continuously by the subject during the selected time period.

42. The method of claim 41, wherein the monitoring patch further comprises verification apparatus to verify that the monitoring patch was worn continuously by the subject during the selected time period.

43. A monitoring patch for monitoring the neural, muscular, and ocular activity of a subject, the monitoring patch comprising:
electroencephalography apparatus to monitor neural activity of the subject during a selected time period, wherein the electroencephalography apparatus comprises an electrode;
electromyography apparatus to monitor muscular activity of the subject during a selected time period, wherein the electromyography apparatus comprises an electrode;
electrooculography apparatus to monitor ocular activity of the subject during a selected period of time, wherein the electrooculography apparatus comprises an electrode;
storage apparatus electrically coupled to the electroencephalography apparatus to store neural activity data, to the electromyography apparatus to store muscular activity data, and to the electrooculography apparatus to store ocular activity data;
a power supply;
adhesive to attach the monitoring patch to the subject; and
wherein the monitoring patch is self-contained.

44. The monitoring patch of claim 43, wherein the electroencephalography apparatus of the monitoring patch samples the neural activity of the subject at about 140 hertz or less.

45. The monitoring patch of claim 43, wherein the electroencephalography apparatus of the monitoring patch monitors the neural activity of the subject that oscillates between about 0.5 hertz to about 70 hertz.

46. The monitoring patch of claim 43, wherein the electromyography apparatus of the monitoring patch samples the muscular activity of the subject at about 140 hertz or less.

47. The monitoring patch of claim 43, wherein the electromyography apparatus of the monitoring patch monitors the muscular activity of the subject that oscillates between about 10 hertz to about 90 hertz.

48. The monitoring patch of claim 43, wherein the electrooculography apparatus of the monitoring patch samples the ocular activity of the subject at about 140 hertz or less.

49. The monitoring patch of claim 43, wherein the electrooculography apparatus of the monitoring patch monitors the ocular activity of the subject that oscillates between about 10 hertz to about 90 hertz.

50. The monitoring patch of claim 43, wherein the storage apparatus of the monitoring patch comprises a removable memory device.

51. The monitoring patch of claim 43, wherein the monitoring patch further comprises an output interface to transmit the neural activity data, the muscular activity data, and the ocular activity data.

52. The monitoring patch of claim 43, wherein the monitoring patch further comprises an indicator to indicate the state of the monitoring patch.

53. The monitoring patch of claim 43, wherein the monitoring patch further comprises verification apparatus to verify that the monitoring patch was worn continuously by the subject during the selected time period.
